(19) United States
(12) Patent Application Publication
(10) Pub. No.: US 2010/0099133 A1
(43) Pub. Date: Apr. 22, 2010

(54) METHOD AND APPARATUS FOR THE TREATMENT OF SPECIMENS

(75) Inventors: Markus Egle, Ladenburg (DE); Jürgen Tenhaef, Brombachtal (DE); Simon Keimer, Nussloch (DE); Karl-Heinz Westerhoff, Eppingen-Elsenz (DE)

Correspondence Address: ALEXANDER R SCHLEE;SCHLEE IP INTERNATIONAL P.C., 3770 HIGHLAND AVENUE, SUITE 203, MANHATTAN BEACH, CA, 90266, US

(73) Assignee: LEICA BIOSYSTEMS NUSSLOCH GMBH, Nussloch (DE)

(21) Appl. No.: 12/578,228

(22) Filed: Oct. 13, 2009

(30) Foreign Application Priority Data
Oct. 22, 2008 (DE) 10 2008 052 659.2
Nov. 10, 2008 (DE) 10 2008 056 584.9

(51) Int. Cl. C12Q 1/02; C12M 1/34

(52) U.S. Cl. 435/29; 435/287.1

(57) ABSTRACT

A method and an apparatus for the treatment of cytological and histological specimens are described. A treatment program comprising multiple treatment parameters is predetermined. The specimens are introduced in accordance with the predetermined treatment program sequentially into a plurality of treatment stations by means of a transport device. At least one of the process data time and number of specimens subject to simultaneous treatment in one particular treatment station and number of the treatment stations during execution of the treatment program are determined. These process data are evaluated for optimizing the treatment program as to at least one of processing time, number of treatment stations and sequence of treatment stations.

### CROSS-REFERENCE TO RELATED APPLICATION

This application claims the priority of the German patent application DE 102008052659.2 having a filing date of Oct. 22, 2008 and of the of the German patent application DE 102008056584.9 having a filing date of Nov. 10, 2008. The entire content of these two prior German patent applications is herewith incorporated by reference.

### BACKGROUND OF THE INVENTION

The invention relates to a method and an apparatus for the treatment of specimens, in particular of cytological and histological prepared specimens, which are preferably arranged on specimen slides and in specimen slide magazines.

Cytological and histological methods are used for the investigation of cells that have been obtained, for example, by way of a smear, or from surgical preparations, biopsy material, or other tissue samples. An analysis of the prepared specimens is performed usually for diagnostic purposes in medicine. The preparation, processing, and staining of such prepared specimens is associated, in most cases, with complex processing of the material using a plurality of reagents. Techniques for identifying tissue that has been modified as a result of illness, for example, require an entire series of steps. These include producing a tissue section, encompassing fixing, dehydration, embedding, and sectioning of the tissue, as well as deparaffination, rehydration, and staining of tissue sections and coverslipping of the completed prepared specimens. Typical reagents that are utilized are formalin, butanol, xylene, and paraffin. Dyes such as hematoxylin, methylene blue, Azur, cresyl violet, toluidine blue, Alcian blue, eosin, azocarmine, acid fuchsin, Ponceau, Orange G, picric acid, or Schiff reagent are also used. Additionally utilized, for the identification and histological localization of substances, are antibodies that are responsible for an antigen-antibody reaction. The antibodies can in turn be detected directly or indirectly by way of certain color reactions in the prepared specimen.

Apparatuses for treatment of the specimens, in particular of cytological and histological prepared specimens, comprise multiple treatment stations that serve for the processing of cells, tissues, or organs. These also include, inter alia, staining. The treatment stations are arranged in multiple rows next to one another. The arrangement is usually fixed. Exchanging or shifting of the treatment stations is, however, possible. In order to transport, treat, and store the specimens, each specimen is arranged on a carrier, in particular on a specimen slide. Multiple specimen slides that are subjected to the same treatment can be arranged in a specimen slide magazine. The treatment stations are equipped with different reagents. Transport is accomplished by means of a transport device from one treatment station to the next. Treatment execution in the various treatment stations is defined by a treatment program. Treatment parameters such as, for example, the nature and sequence of the treatment stations and the treatment duration for each treatment station, are constituents of a treatment program. The treatment program with its treatment parameters, and the position of the treatment stations in the apparatus, are defined by an operator or a user on the basis of his or her experience.

It turns out to be disadvantageous that the user's experience is often not sufficient to optimize a treatment in terms of its quality, the total duration of treatment, the number of samples to be treated, and the prioritization of individual additional samples. For example, if the residence times in individual treatment stations are longer than in other treatment stations, this results in a buildup of specimens before the individual treatment stations, and in corresponding waiting times. The individual treatment stations represent a bottleneck in the overall treatment. Waiting times of this kind can result in different treatment results depending on the length of the waiting time. The treatment result is thus no longer reproducible, but instead depends on the particular bottlenecks in the context of treatment. If the treatment involves, for example, staining, an excessive standing time in a water station can cause the stain to be washed out.

### SUMMARY OF THE INVENTION

The object underlying the invention is that of making available a method and an apparatus for the treatment of specimens that enable automatic and continuous improvement of a treatment program in order to improve the treatment quality, shorten the treatment duration, and enable the simultaneous treatment of multiple specimens.

This is achieved according to the invention by a method for the treatment of cytological and histological specimens comprising: predetermining a treatment program comprising multiple treatment parameters for treatment of the specimens; introducing the specimens in accordance with the predetermined treatment program sequentially into a plurality of treatment stations by means of a transport device; determining at least one of the process data time and number of specimens subject to simultaneous treatment in one particular treatment station and number of the treatment stations during execution of the treatment program; evaluating the determined process data after the execution of each treatment program; and optimizing the treatment program as to at least one of processing time, number of treatment stations and sequence of treatment stations.

According to the invention, this method can be performed by an apparatus for the treatment of cytological and histological prepared specimens, comprising: multiple treatment stations into which the specimens are introduced in accordance with a predefined treatment program; a transport device that is adapted to introduce the specimens into the individual treatment stations and to transport the specimens from one treatment station to the next treatment station; at least one sensor for reading process data relevant to at least one of processing time, number of treatment stations and sequence of treatment stations during execution of the treatment program; and a device configured to evaluate the read process data after the execution of each treatment program and to optimize the treatment program with regard to at least one of processing time, number of treatment stations and sequence of treatment stations.

The method according to the present invention is notable for the fact that the process data relevant to time and/or number with relation to the specimens and/or the treatment stations are sensed during execution of the treatment program. Included thereamong are, for example, the time during which a specimen is present in the individual treatment stations, optionally a waiting time before a specimen can be introduced into a treatment station because the latter is otherwise occupied, the total duration of treatment in all the treatment stations involved in treatment, the sum of the waiting times before all the treatment stations of a treatment, and the starting time of a treatment. With regard to number, what is sensed is, for example, the number of specimens per treatment, the throughput for each treatment station, and the volume of samples or specimens. Serving as sensors for sensing the process data are, for example, a time sensing device, a light barrier, or other devices for sensing specific events. The sensed process data are then evaluated. The result of the evaluation supplies, for example, an information item as to those treatment stations at which a bottleneck exists, and where particularly long waiting times therefore occur. It is furthermore possible to ascertain, for example, which of the treatment stations is being used seldom or not at all. It is further possible to obtain, from the time that elapses between the individual treatments without occurrence of a waiting time, an information item as to the length of the distance that a specimen must travel from one treatment station to the next. Lastly, an optimization for the treatment program and/or the treatment stations is determined from the evaluation. An optimization consists, for example, in increasing or reducing the number of specific treatment stations, or changing the position of the treatment stations, as a function of the evaluation.

### DETAILED DESCRIPTION OF THE INVENTION

An advantage of the method according to the present invention is that the treatment program and/or the treatment stations are not adapted only once to changed requirements, but that a continuous and automatic optimization is accomplished. The method therefore offers the capability of reacting continuously to changes, for example to an increased or reduced volume of specimens. The optimization is based not on the experience of an operator or user, but instead on a continuous analysis of the process data obtained during execution of the method. The process that occurs in this context is a self-teaching process. The method can thus react even to short-term changes and optimize the treatment sequence.

A further advantage of the method according to the present invention consists in prioritizing a specific specimen and carrying out treatment of that specimen in preferred fashion. Individual specimens for which the result of a treatment is required particularly quickly can be treated in parallel with other specimens with no need to interrupt the treatment of those other specimens for that purpose. For this, the two treatments proceeding in parallel are coordinated in such a way that the shortest possible waiting times occur before the individual treatment stations, and the quality of the samples is not impaired. Treatment of the individual specimen can be started while the treatment of other specimens is still ongoing.

The samples are often unique items that represent a certain value. It is therefore very important that the quality of the specimens not be impaired during treatment, and that a reproducible result be achieved by means of the treatment. The method according to the present invention takes this into account, and meets the requirements necessary therefor. In addition, the treatment stations filled with expensive reagents are protected, by means of the method, from avoidable contamination. A high-quality treatment is thereby guaranteed. In addition, the reagents require infrequent replacement, which promotes environmental protection. Lastly, the treatment is carried out as rapidly as possible in consideration of the predefined parameters. The result of the treatment can thus be presented as quickly as possible.

According to an advantageous embodiment of the invention, the optimization is outputted to the user. Provided for this purpose is, for example, an optical display device on which the recommendation regarding optimization is displayed. In this fashion the user receives, for example, the information item to increase or reduce the number of specific treatment stations, or to change the position of individual treatment stations.

The user can then carry out, him- or herself, the changes necessary for optimization. The possibility furthermore exists that the treatment program on which the treatment is based can be automatically coordinated with the optimization. These include, for example, optimizations relevant to the sequence of specific treatment stations in the treatment program.

According to a further advantageous embodiment of the invention, the treatment program is automatically modified in accordance with the optimization for the treatment of specimens subsequent to the determination of the optimization.

According to a further advantageous embodiment of the invention, the waiting time before a treatment station, the transit time through all the treatment stations, the process duration of the entire treatment, and/or the starting time of a treatment are sensed as process data. These process data are relevant to time in relation to the treatment and/or in relation to the individual treatment stations.

According to a further advantageous embodiment of the invention, the number of specimens in a treatment station, the number of specimens in a specimen slide magazine, the frequency with which a specimen is introduced into a treatment station during a treatment, and/or the throughput per treatment station, are sensed as process data. These process data are relevant not to time but to the specimens and the treatment stations. These process data are detected by means of a light barrier or another sensor for sensing an event, or are derived automatically from the treatment program. The data relevant to time and the data relevant to number can also be combined with one another.

According to a further advantageous embodiment of the invention, the lengths of the distances traveled between two treatment stations into which the specimen is successively introduced in accordance with the treatment program are sensed as process data. This distance can be ascertained, for example, by means of a transducer or a sensor on the transport device. In addition, the length of the travel distance can be calculated from the positions of the treatment stations, if they are known. A certain amount of information as to the length of the travel distance is also supplied by the time elapsing between removal of a specimen from a first treatment station and introduction of the specimen into the second treatment station. It is, however, difficult to separate the time required by the specimen to travel the distance between the two treatment stations from a possible waiting time before the second station. Determination of the distance actually traveled therefore supplies a more accurate result.

According to a further advantageous embodiment of the invention, the fill level for each of the treatment stations filled with a liquid treatment bath is determined. An information item is thereby continuously available with regard to the reagents, in particular the quantity of the reagents, contained in the treatment stations. The user can be continuously informed as to the fill level. The possibility furthermore exists of outputting a warning to the user when the fill level falls below a predefined limit.

According to a further advantageous embodiment of the invention, the treatment program contains multiple treatment parameters, among which are included the sequence in which a specimen is introduced into the various treatment stations, and the treatment duration for each treatment station. Further treatment parameters can likewise be defined, and represent a constituent of the treatment program. In contrast to the process data, the treatment parameters are defined together with the treatment.

According to a further advantageous embodiment of the invention, a determination is made, for evaluation purposes, of those treatment stations into which no specimens are introduced during a treatment.

According to a further advantageous embodiment of the invention, a determination is made, for evaluation purposes, of those treatment stations into which specimens are most often introduced. Advantageously, for the optimization those treatment stations into which no specimens are introduced are replaced by treatment stations into which specimens are most often introduced. The user then receives a recommendation to exchange the corresponding treatment stations with one another.

According to a further advantageous embodiment of the invention, for the optimization a number and an arrangement of treatment stations for which the waiting times between the treatment stations are minimal is determined. A minimal waiting time shortens the overall processing or treatment duration without thereby impairing the quality of the sample or specimen.

According to a further advantageous embodiment of the invention, for the optimization a number and an arrangement of treatment stations that enable simultaneous treatment of as many specimens as possible is determined. It is thereby possible to treat not just one specimen, but multiple specimens in parallel with a treatment that is already in progress. This makes possible the treatment of one or more specimens without, for that purpose, interrupting the treatments of the individual specimens. It is thus possible to treat specific specimens in preferred fashion without impairing other specimens, already being treated, in terms of their treatment or their quality. The treatments can proceed according to the same treatment program or according to different treatment programs.

According to a further advantageous embodiment of the invention, each specimen is equipped, before treatment, with an individual machine-readable marking. The marking can be applied directly on the specimen, on the specimen slide, or on the specimen slide magazine. The marking is sensed before the treatment begins and/or at all or at individual treatment stations and/or at the conclusion of the treatment. It is thereby possible to interrogate and check the current treatment station or another position of the specimen at any time during a treatment. This is also referred to as “data tracking.”

According to a further advantageous embodiment of the invention, the treatment stations are treatment stations, provided with reagents, of an automatic staining machine. An automatic staining machine of this kind is also referred to as a “stainer.”

The treatment stations of a stainer are typically stations provided with reagents, water stations, heating stations or ovens, unloading stations, and loading stations.

The apparatus according to the present invention for the treatment of specimens, in particular of cytological and histological prepared specimens, which preferably are arranged on specimen slides and in specimen slide magazines, is notable for the fact that it is equipped with at least one sensor for sensing process data relevant to time and/or number in relation to the specimens and/or the treatment stations during execution of a treatment program. The sensor senses, for example the time during which a specimen is present in the individual treatment stations, optionally a waiting time before a specimen can be introduced into a treatment station, the total duration of treatment in all the treatment stations involved in the treatment, the sum of the waiting times before all treatment stations of a treatment, and the starting time of a treatment. With regard to number, for example, the number of specimens per treatment is sensed, the throughput per treatment station, and the volume of samples or specimens. A time sensing device, for example a clock, serves for time sensing. Time sensing is triggered by a specific event. This event is either likewise sensed by a sensor, or is predefined by the treatment program. An event represents, for example, the introduction of a specimen into a treatment station or the removal of a specimen from a treatment station. In addition, the apparatus for the treatment of specimens is equipped with a device for evaluating the sensed process data and for optimizing the treatment program and/or the treatment stations. The device for evaluation and optimization can be, for example, a calculator or a computer. The latter is equipped with a software program suitable for comparing the process data to predefined guidelines. The apparatus can furthermore calculate independent guidelines from modified arrangements of treatment stations.

The apparatus serves, in particular, to carry out the method according to the present invention.

According to a further advantageous embodiment of the apparatus according to the present invention, the latter is equipped with a device for sensing machine-readable markings. These markings are arranged on the specimens, on the specimen slides, or on the specimen slide magazines. It is thereby possible to ascertain, at any time during treatment, the treatment station in which a specimen is currently located. The device or devices for sensing machine-readable markings are arranged for this purpose on multiple, or on all, treatment stations.

Further advantages and advantageous embodiments of the invention may be gathered from the description below, from the drawings, and from the claims.

### BRIEF DESCRIPTION OF THE DRAWINGS

The drawings depict an exemplifying embodiment of an apparatus according to the present invention. In the drawings:

FIG. 1 is a front view of an apparatus;

FIG. 2 is a view from the side of the apparatus in accordance with FIG. 1;

FIG. 3 shows a specimen slide magazine, having multiple specimen slides, of the apparatus in accordance with FIG. 1;

FIG. 4 shows a computer and sensor of the apparatus in accordance with FIG. 1.

### DETAILED DESCRIPTION OF THE DRAWINGS

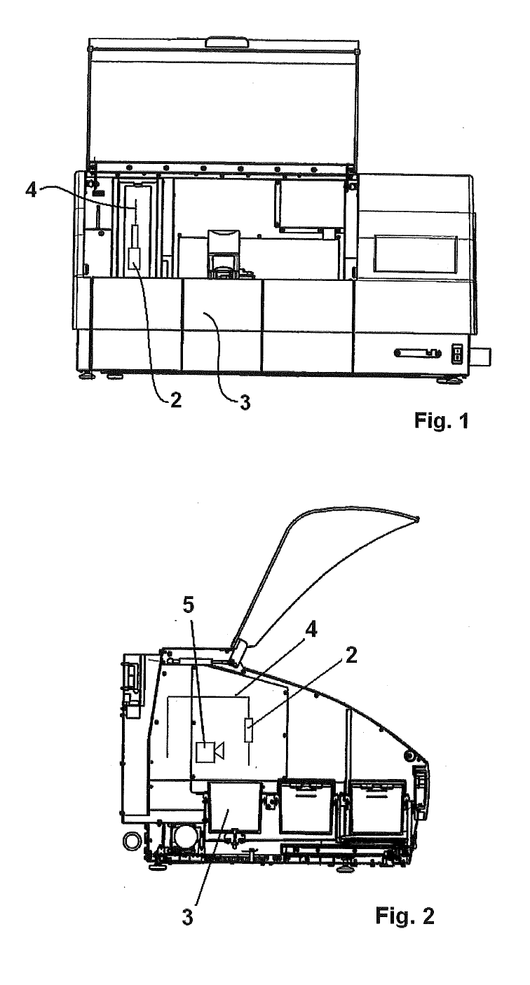

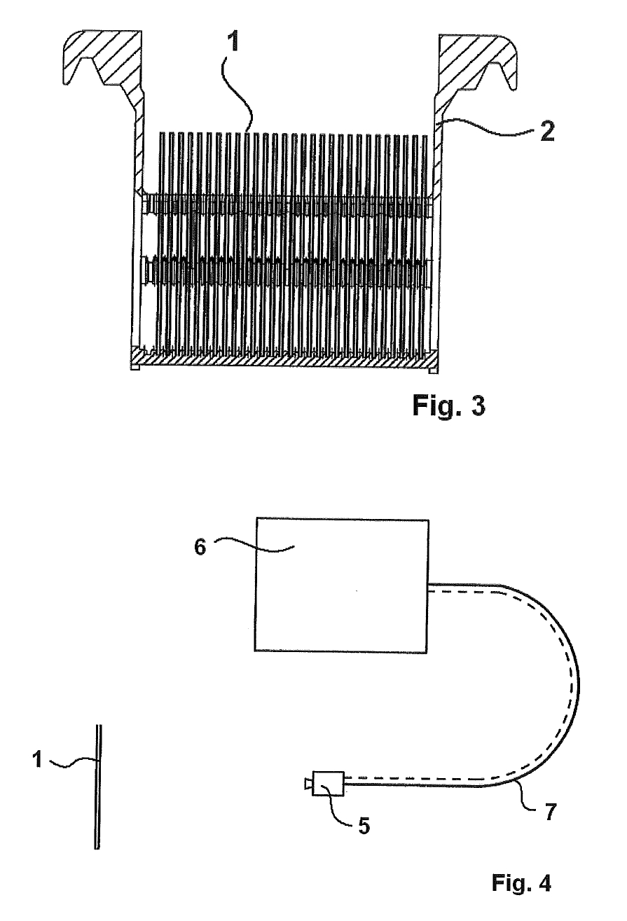

FIGS. 1 and 2 depict an apparatus for the treatment of specimens, in particular of cytological and histological prepared specimens, the specimens being arranged on specimen slides 1. Specimen slides 1 are arranged in specimen slide magazines 2 for treatment in the apparatus. The arrangement of multiple specimen slides 1 in a specimen slide magazine is depicted in FIG. 3. The apparatus according to FIGS. 1 and 2 comprises multiple treatment stations 3 into which the specimens arranged on specimen slides 1 are introduced according to a predefined treatment program. Treatment stations 3 are located next to one in the apparatus. They are filled with a variety of treatment baths. A transport device 4 is arranged in the apparatus above the treatment stations. It engages onto specimen slide magazines 2 from above, picks up the specimen slide magazines, and transports them from one treatment station 3 to the next. In addition, transport device 4 picks up a specimen slide magazine at the beginning of a treatment in order to introduce it into first treatment station 3. Transport device 4 furthermore conveys a specimen slide magazine out of the apparatus after the treatment is complete and the last treatment station has been left.

The apparatus is equipped with an optical sensor 5 with which markings on specimen slides 1 and on specimen slide magazine 2 can be sensed. The data sensed with sensor 5 are evaluated with a computer 6. Sensor 5 is connected to computer 6 via a data line 7. Sensor 5, computer 6, and data line 7 are depicted, together with a specimen slide, in FIG. 4.

All the features, both individually and in any combination with one another, may be essential to the invention.

### LIST OF REFERENCE NUMERALS

1 Specimen slide

2 Specimen slide magazine

3 Treatment station

4 Transport device

5 Sensor

6 Computer

7 Data line

What is claimed is:

1. A method for the treatment of at least one of cytological and histological specimens comprising:
predetermining a treatment program comprising multiple treatment parameters for treatment of the specimens;
introducing the specimens in accordance with the predetermined treatment program sequentially into a plurality of treatment stations by means of a transport device;
determining at least one of the process data time and number of specimens subject to simultaneous treatment in one particular treatment station and number of the treatment stations during execution of the treatment program;
evaluating the determined process data after the execution of each treatment program; and
optimizing the treatment program as to at least one of processing time, number of treatment stations and sequence of treatment stations.

2. The method according to claim 1, further comprising displaying the optimization to a user.

3. The method according to claim 1, further comprising modifying the treatment program for the subsequent treatment of specimens in accordance with the optimization.

4. The method according to claim 1, further comprising determining as process data at least one of the waiting time prior to treatment in a treatment station, the transit time through all the treatment stations, the process duration of the entire treatment, and the starting time of a treatment.

5. The method according to claim 1, further comprising determining as process data the number of specimens in a treatment station, the number of specimens in a specimen slide magazine, the frequency of introducing a specimen into a treatment station during a treatment, and the throughput per treatment station.

6. The method according to claim 1, further comprising determining as process data a specimen traveling distance between two subsequent treatment stations into which the specimen is introduced in sequence in accordance with a treatment program.

7. The method according to claim 1, further comprising determining as process data the fill level of each one of the treatment stations that are filled with a liquid treatment bath.

8. The method according to claim 1, further comprising providing as treatment parameters the sequence of the treatment stations and the treatment duration for each treatment station as part of the treatment program.

9. The method according to claim 1, further comprising determining for evaluation purposes those treatment stations into which no specimens are introduced.

10. The method according to claim 1, further comprising determining for evaluation purposes those treatment stations into which specimens are most frequently introduced.

11. The method according to claim 8, further comprising determining for evaluation purposes those treatment stations into which no specimens are introduced and those treatment stations into which specimens are most frequently introduced and replacing those treatment stations into which no specimens are introduced by treatment stations into which specimens are most frequently introduced.

12. The method according to claim 1, further comprising determining for the optimization a number and an arrangement of treatment stations for which the waiting times between the treatment stations are minimal.

13. The method according to claim 1, further comprising determining for the optimization a number and an arrangement of treatment stations that enables simultaneous treatment of as many specimens as possible.

14. The method according to claim 1, further comprising determining for the optimization a number and an arrangement of treatment stations that enable the treatment of an individual specimen during the treatment of one or more other specimens without interrupting treatments for that purpose.

15. The method according to claim 1, further comprising providing each specimen prior to treatment with an individual machine-readable tag; and reading the tag at least at one point in time prior to beginning the treatment, at all or at individual treatment stations and at the conclusion of the treatment.

16. The method according to claim 1, further comprising providing the treatment stations with reagents of an automatic staining machine.

17. The method according to claim 1, further comprising placing a plurality of specimens on respective specimens slide and placing the specimen slides in at least one specimen slide magazine.

18. An apparatus for the treatment of at least one of cytological and histological prepared specimens, comprising:
multiple treatment stations into which the specimens are introduced in accordance with a predefined treatment program;
a transport device that is adapted to introduce the specimens into the individual ones of the treatment stations and to transport the specimens from one treatment station to the next treatment station;
at least one sensor for reading process data relevant to at least one of processing time, number of treatment stations and sequence of treatment stations during execution of the treatment program; and
a device configured to evaluate the read process data after the execution of each treatment program and to optimize the treatment program with regard to at least one of processing time, number of treatment stations and sequence of treatment stations.

19. The apparatus according to claim 18, wherein the sensor is a time sensing device.

20. The apparatus according to claim 18, wherein the sensor is a device for reading machine-readable tags.

21. The apparatus according to claim 18, wherein the device configured to evaluate the process data is a computer that is connected to the at least one sensor via an interface.

22. The apparatus according to claim 18, further comprising specimen slides for holding the specimens and at least one specimen slide magazine for holding a plurality of specimen slides.
